(19) United States
(12) Patent Application Publication
(10) Pub. No.: US 2024/0009086 A1
(43) Pub. Date: Jan. 11, 2024

(54) DEVICES, SYSTEMS, AND METHODS FOR SECURING TREATMENT MEMBERS

(71) Applicant: Phagenesis Limited, Manchester (GB)

(72) Inventor: Conor Mulrooney, Manchester (GB)

(21) Appl. No.: 18/253,525

(22) PCT Filed: Nov. 19, 2021
(86) PCT No.: PCT/GB2021/053008

§ 371 Date: May 18, 2023

Related U.S. Application Data
(60) Provisional application No. 63/198,911, filed on Nov. 20, 2020.

(51) Int. Cl. A61J 15/00

(52) U.S. Cl. CPC A61J 15/0061; A61J 15/0003

(57) ABSTRACT

Devices for securing an elongated treatment member to a patient and associated systems and methods are disclosed herein. In some embodiments, the device is configured to secure a medical device comprising a nasally-inserted or an orally-inserted elongated treatment member to a face of a patient. The device can comprise one or more portions configured to be adhered to the patients face and one or more portions configured to secure a length of an extracorporeal portion of the elongated treatment member to the device. The device can comprise an adhesive forming an adhesive bond with the patients skin and the adhesive bond can have an adhesive bond strength sufficient to prevent the patient from prematurely removing the device or the treatment member. A releasing agent can be applied to the device to degrade the adhesive bond strength between the device and the patients skin such that the device can be removed from the patients face without substantial discomfort to the patient.

### CROSS REFERENCE TO RELATED APPLICATION(S)

The present application claims the benefit of priority to U.S. Provisional Patent Application No. 63/198,911, titled DEVICES, SYSTEMS, AND METHODS FOR SECURING TREATMENT MEMBERS, filed Nov. 20, 2020, which is incorporated by reference herein in its entirety.

### TECHNICAL FIELD

The present disclosure is directed generally to devices, systems, and methods for securing a treatment member to a patient. Particular embodiments include a device for securing an extracorporeal portion of a nasally-inserted elongated treatment member, such as a nasogastric or nasoenteric tube, to a face of a patient.

### BACKGROUND

To maintain nutrition and hydration in patients with an unsafe swallow, particularly in the hospital setting, it is standard practice to insert a nasogastric feeding tube to allow material to be safely delivered directly to the stomach. These tubes stay in place for up to 4 to 6 weeks. Thereafter the tubes are either removed because the patient's swallow status has improved sufficiently, or if it has not, are removed and replaced instead with a gastrostomy tube which is more suited to longer term feeding.

Swallowing dysfunction requiring enteral nutrition delivery via a nasogastric tube is very common after brain injury due to stroke, trauma, or infection, or because of prolonged periods of mechanical ventilation. Ensuring the patient receives adequate nutrition is critical in the immediate period after injury or ventilation to maximize recovery and rehabilitation.

It is not uncommon for patients, particularly after brain injury, to be confused and/or combative. Nasogastric feeding tubes, while effective in supporting nutritional needs, are generally uncomfortable for the patient. Patients often try to pull out the feeding tubes as a result. Replacing and retaining the feeding tubes can be labor intensive and upsetting for the patient.

To address the foregoing concerns, low tack medical tape 100 is commonly used to secure a nasogastric tube 101 to a patient, as shown in FIG. 1. The medical tape must be low tack so that the tape can later be removed with minimal pain to the patient and without injuring the patient's skin. The latter consideration is particularly important for certain patient populations known to have fragile skin, such as elderly patients. However, the low adhesion properties of low tack tape allow patients to remove the tape with relative ease and with relatively little discomfort. Thus, low tack medical tape is not a sufficient deterrent for nasogastric tube removal.

Another device used for securing nasogastric tubes is a nasal bridle 200, for example as shown in FIG. 2. A looped portion of the bridle 200 extends through one nostril, around the nasal septum, and back through the other nostril. The ends of the loop extend out of the nostrils and are secured together at a location external of the patient's nasal cavity. For example, as shown in FIG. 2, the nasal bridle 200 can include a clip positioned external to the patient's nasal cavity and configured to retain the ends of the loop of the bridle 200. The ends of the loop can be secured to the nasogastric tube such that the nasogastric tube is indirectly secured to the patient's septum via the loop. While the nasal bridle 200 is associated with lower rates of tube dislodgement than medical tape, bridle insertion is traumatic for the patient and the bridle itself is extremely uncomfortable once installed. Moreover, a patient attempting to remove a feeding tube 201 secured with a nasal bridle 200 can tear their septum in the process. For example, some patients with brain injury have compromised sensory systems and may not realize that pulling on the nasal bridle and/or feeding tube stresses their nasal septum and may tear through their nasal septum.

In view of the above there is a need for devices, systems, and methods to retain and secure nasally inserted catheters or other elongated treatment members in a manner that is minimally traumatic for the patient and safe and easy for healthcare workers to use.

### SUMMARY

The present technology relates to devices, systems, and methods for securing a treatment member relative to a patient. In particular embodiments, the present technology comprises a device configured to secure an extracorporeal portion of a nasally- or orally-inserted elongated treatment member (e.g., a catheter, a feeding tube, etc.) to a face of a patient to prevent or substantially inhibit displacement or removal of the device and associated treatment member. Several embodiments of the present disclosure, for example, are configured to secure a nasally-inserted enteral feeding tube, such as a nasoenteric tube or a nasogastric tube, to a face of a patient. The subject technology is illustrated, for example, according to various aspects described below, including with reference to FIGS. 1-7. Various examples of aspects of the subject technology are described as numbered clauses (1, 2, 3, etc.) for convenience. These are provided as examples and do not limit the subject technology.
1. A device configured to secure a position of an extracorporeal portion of an elongated treatment member inserted into a body lumen of a patient, the device comprising:
a first portion configured to be adhered to a nose of the patient;
a second portion extending from the first portion along a longitudinal dimension of the device, wherein the second portion is configured to be adhered to a cheek of the patient; and
a third portion extending from the second portion in a direction laterally away from the longitudinal dimension of the device, wherein the third portion is configured to be folded over a length of the extracorporeal portion of the elongated treatment member and secured to the device such that the elongated treatment member is secured to the device,
wherein each of the first and second portions includes an adhesive, and
wherein each of the first, second, and third portions comprise a unitary structure.
2. The device of Clause 1, wherein the adhesive has an adhesion strength sufficiently large to prevent the device from being mechanically removed from the patient's face without discomfort.
3. The device of Clause 1 or Clause 2, wherein, when a releasing agent is applied to the device, the releasing agent reduces the adhesion strength of the adhesive such that the device can be mechanically removed from the patient's face.
4. The device of any one of Clauses 1 to 3, wherein the elongated treatment member comprises a nasogastric tube.
5. The device of any one of Clauses 1 to 4, further comprising a first material and a second material, wherein the first and second materials are coextensive with the first and second portions, wherein the adhesive is positioned between the first and second materials.
6. The device of Clause 5, wherein the first material is configured to face away from the patient's face when the device is adhered to the patient's face.
7. The device of Clause 5 or Clause 6, wherein the second material is configured to be removed prior to adhering the device to the patient's face.
8. The device of any one of Clauses 5 to 7, wherein the first material comprises a breathable material and the second material comprises a backing paper.
9. The device of any one of Clauses 1 to 8, wherein:
the third portion comprises a first side and a second side opposite the first side along a thickness of the third portion, and, when the first and second portions are adhered to the patient's face and the third portion has not yet been folded over the elongated treatment member, the second side is positioned proximate the patient's face,
wherein the device further comprises a removable element and an adhesive element positioned on the first side of the third portion, wherein the adhesive element is positioned between the removable element and the first side.
10. The device of Clause 9, wherein the removable element is backing paper configured to be removed from the device to expose the adhesive element.
11. The device of Clause 9 or Clause 10, wherein the adhesive element comprises an absorbent material.
12. The device of any one of Clauses 1 to 11, wherein the first portion includes a resilient member configured to conform the first portion to the patient's nose.
13. The device of any one of Clauses 1 to 12, wherein the second portion of the device comprises one or more openings configured to receive the elongated treatment member such that the elongated treatment member extends along the longitudinal dimension of the device.
14. The device of any one of Clauses 1 to 13, further comprising a channel extending along at least a portion of the first portion and at least a portion of the second portion, wherein the portion of the device defining the channel does not include the adhesive, and wherein the channel is configured to receive a releasing agent therethrough to enable mechanical separation of the device from the patient's skin.
15. The device of Clause 14, further comprising a valve fluidly coupled to the channel and configured to receive the releasing agent from an external source.
16. The device of any one of Clauses 1 to 15, wherein the adhesive is a high tack adhesive.
17. The device of any one of Clauses 1 to 16, wherein the first portion has a surface area sufficiently large such that the first and second portions are configured to prevent the device from being mechanically removed from the patient's face without discomfort.
18. A device configured to secure an extracorporeal portion of an elongated treatment member inserted into a body lumen of a patient to a face of a patient, the device comprising:
a nasal portion comprising a first surface including an adhesive and a second surface opposite the first surface along a thickness of the nasal portion, the first surface of the nasal portion being configured to be adhered to a dorsum nasi of the patient, wherein the nasal portion has a first end configured to be adhered to a first ala region of the patient and a second end configured to be adhered to a second ala region of the patient;
a facial portion comprising a first surface including an adhesive and a second surface opposite the first surface along a thickness of the facial portion, the first surface of the facial portion being configured to be adhered to a cheek of the patient, wherein the facial portion has a first end at the nasal portion and a second end spaced apart from the first end along a first direction; and
a securing portion comprising a first surface and a second surface opposite the first surface along a thickness of the securing portion, wherein the securing portion has a first end at the facial portion and a second end spaced apart from the first end along a second direction disposed at an angle with respect to the first direction,
wherein, the securing portion comprises a first state in which the first surface is configured to be positioned proximate the patient's face and a second state in which the securing portion is configured to be folded over the extracorporeal portion of the elongated treatment member such that the second surface is secured to the device and a position of the extracorporeal portion of the elongated treatment member is fixed with respect to the device, and
wherein the second surface of the nasal portion, the second surface of the facial portion, and the first surface of the securing portion comprise a monolithic structure.
19. The device of Clause 18, wherein, when the nasal portion is adhered to the patient's dorsum nasi and the facial portion is adhered to the patient's cheek, an adhesive bond strength between the device and the patient's face prevents the device from being mechanically separated from the patient's face without discomfort to the patient.
20. The device of Clause 19, wherein, when a releasing agent is applied to the device, the adhesive bond strength between the device and the patient's face is reduced such that the device can be mechanically separated from the patient's face without discomfort to the patient.
21. The device of any one of Clauses 18 to 20, wherein each of the second surface of the nasal portion, the second surface of the facial portion, and the first surface of the securing portion comprises a breathable material.
22. The device of any one of Clauses 18 to 21, wherein, the first direction substantially corresponds to a longitudinal dimension of the device.
23. The device of any one of Clauses 18 to 21, wherein, when the device is adhered to the patient's face, the first direction extends from the first end to the second end along a substantially inferior direction.
24. The device of any one of Clauses 18 to 23, wherein the elongated treatment member is an electrical stimulation device.
25. The device of any one of Clauses 18 to 24, wherein at least one of the first surface of the nasal portion, the first surface of the facial portion, or the second surface of the securing portion comprises a backing material configured to prevent the device from being adhered to the patient's face until the backing material is removed.
26. The device of any one of Clauses 18 to 25, wherein at least one of the first surface of the nasal portion, the first surface of the facial portion, or the second surface of the securing portion comprises an absorbent material carrying the adhesive.
27. The device of any one of Clauses 18 to 26, wherein the nasal portion comprises a resilient member configured to conform the first portion to the patient's dorsum nasi.
28. The device of any one of Clauses 18 to 27, wherein the facial portion of the device comprises one or more openings configured to receive the elongated treatment member such that, when the device is adhered to the patient's face, the elongated treatment member extends laterally along the patient's cheek.
29. The device of any one of Clauses 18 to 28, further comprising a channel extending along a periphery of the first portion and the second portion, wherein the periphery of the first portion and the second portion does not include the adhesive, and wherein the channel is configured to receive a releasing agent therethrough to enable mechanical separation of the device from the patient's skin.
30. The device of Clause 29, further comprising a port fluidly coupled to the channel and configured to receive the releasing agent from an external source.
31. A device configured to secure a position of a proximal portion of an elongated treatment member inserted into a body lumen of a patient, the device comprising:
a first broad surface comprising a first shape including a first portion, a second portion, and a third portion; and
a second broad surface opposite the first broad surface along a thickness of the device, the second broad surface comprising a second shape including a first portion, a second portion, and a third portion, wherein the second shape is substantially the same as the first shape,
wherein the first and second portions of the first broad surface and the third portion of the second broad surface comprise an absorbent material with an adhesive disposed thereon,
wherein the third portion of the first broad surface and the first and second portions of the second broad surface comprise a breathable material,
wherein the first and second portions of the first broad surface are configured to be adhered to a nose of the patient and a face of the patient, respectively, and
wherein the third portion of the second broad surface is configured to be adhered to the proximal portion of the elongated treatment member.
32. The device of Clause 31, wherein the first and second portions of the first broad surface have a surface area sufficiently large to prevent the patient from prematurely removing the device from the patient's face.
33. The device of Clause 31 or Clause 32, wherein the device is configured to receive a solvent configured to dissolve the adhesive such that the device can be removed from the patient's face without discomfort to the patient.
34. The device any one of Clauses 31 to 33, wherein the breathable material is opaque and the adhesive is light-sensitive.
35. The device of Clause 34, wherein the breathable material is configured to be removed from the device such that the adhesive is exposed to light and dissolves such that the device can be removed from the patient's face without discomfort to the patient.
36. A method of securing an extracorporeal portion of an elongated treatment member inserted into a body lumen of a patient to a device and securing the device to a face of the patient, the method comprising:
adhering a nasal portion of the device to a nose of the patient;
positioning a first region of the extracorporeal portion of the elongated treatment member proximate a securing portion of the device;
folding the securing portion of the device over the first region of the extracorporeal portion of the elongated treatment member and adhering the securing portion to the device;
positioning a second region of the extracorporeal portion of the elongated treatment member within an opening defined by a facial portion of the device; and
adhering the facial portion of the device to a cheek of the patient.
37. The method of Clause 36, wherein the nasal portion is adhered to the patient's nose and the facial portion is adhered to the patient's check with an adhesive bond strength sufficiently high to prevent the device from being mechanically removed from the patient's face.
38. A method of removing a device adhered to a face of a patient via an adhesive, the method comprising:
delivering a releasing agent from an external source to a valve of the device;
delivering the releasing agent from the valve to a channel of the device, wherein the channel extends along at least a portion of the device, the portion of the device being at or near a periphery of the device;
reducing an adhesive bond strength between the device and the patient's face in at least one location proximate the channel; and
applying a force to the device such that the device mechanically separates from the patient's face.
39. A device configured to secure a position of an extracorporeal portion of an elongated treatment member inserted into a body lumen of a patient relative to a face of the patient, the device comprising:
a body comprising:
   a first portion configured to be adhered to a nose of the patient;
   a second portion extending from and continuous with the first portion along a longitudinal dimension of the device, wherein the second portion is configured to be adhered to a cheek and/or jaw of the patient; and
a retaining element at the body, the retaining element configured to detachably couple the extracorporeal portion of the elongated treatment member to the device,
wherein each of the first and second portions includes a high tack adhesive.
40. The device of Clause 39, wherein the retaining element is integral with the body.
41. The device of Clause 39, wherein the retaining element is a separate element fastened to the body.
42. A device configured to secure a position of an extracorporeal portion of an elongated treatment member inserted into a body lumen of a patient, the device comprising:
a first material comprising an adhesive agent configured to adhere the device to the patient's skin;
a second material on the first material, the second material having a perimeter and an area bound by the perimeter;
a plurality of interconnected channels between the first and second materials; and
an access port fluidly coupled to the channels,
wherein, while the device is adhered to the patient's skin: (a) the port is configured to provide fluid access to the channels, and (b) delivery of a releasing agent to the channels via the port distributes the releasing agent across and/or along at least some of the area of the second material, thereby neutralizing the adhesive agent and enabling removal of the device from the patient's skin.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 depicts a prior art medical tape configured to secure an elongated treatment member.

FIG. 2 depicts a prior art nasal bridle configured to secure an elongated treatment member.

FIG. 3 is a top view of a device configured to secure an elongated treatment member to a patient in accordance with several embodiments of the present technology.

FIG. 4 is an exploded view of the device shown in FIG. 3

FIG. 5 depicts a device configured to secure an elongated treatment member to a patient in accordance with several embodiments of the present technology. In FIG. 5, the device is shown adhered to a patient's face.

FIG. 6 depicts a device configured to secure an elongated treatment member to a patient and an external source of a releasing agent in accordance with several embodiments of the present technology.

FIG. 7 depicts a device configured to secure an elongated treatment member to a patient in accordance with several embodiments of the present technology. In FIG. 7, the device is shown adhered to a patient's face.

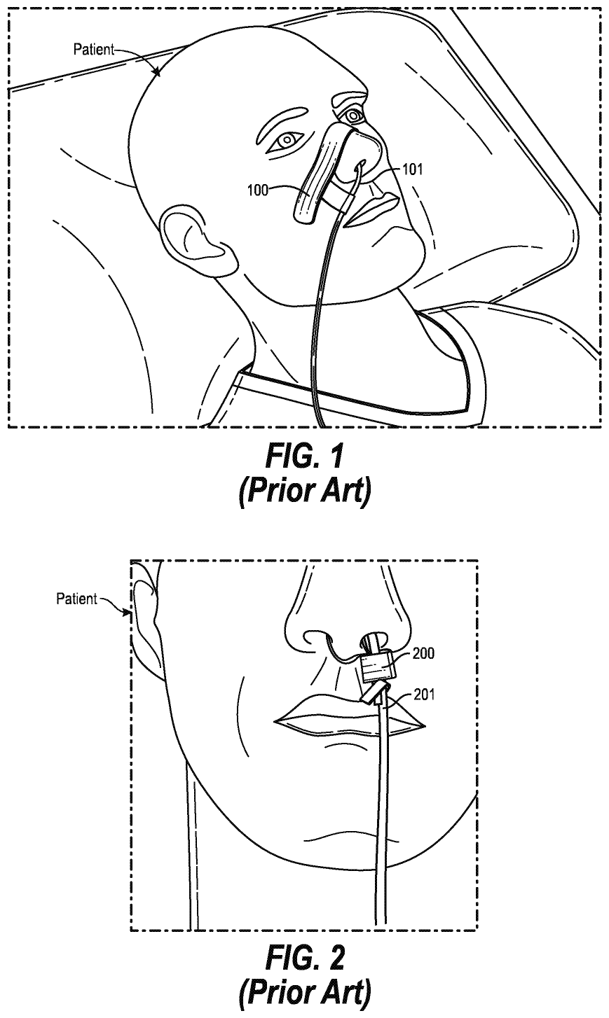

### DETAILED DESCRIPTION

The devices disclosed herein are configured to address the limitations associated with conventional methods of securing elongated treatment members that have been partially inserted into a body lumen of a patient. In some embodiments, the present technology includes a device (such as a medical dressing) configured to secure an elongated treatment member (e.g., a nasogastric tube, a catheter, etc.) to the patient's face. The device can be configured to be adhered to the patient's face via an adhesive. According to several embodiments, the device has a large surface area and is configured to adhere to the patient's face with a high adhesive bond strength. These features of the device, alone or in combination, cause the device to be strongly adhered to the patient's face such that the patient cannot remove the device without discomfort. As such, the device is configured to disincentivize combative and/or confused patients from prematurely removing or dislodging the treatment member and/or device. When a clinician determines that the device and treatment member should be removed, the device is configured to be detached from the patient's face without discomfort by employing a selective release mechanism to deactivate and/or weaken the adhesive bond between the device and the patient's skin. For example, the clinician can apply a solvent to the device that is configured to dissolve the adhesive. In some embodiments, the adhesive is light-sensitive and the clinician can expose the adhesive to light to degrade the adhesive.

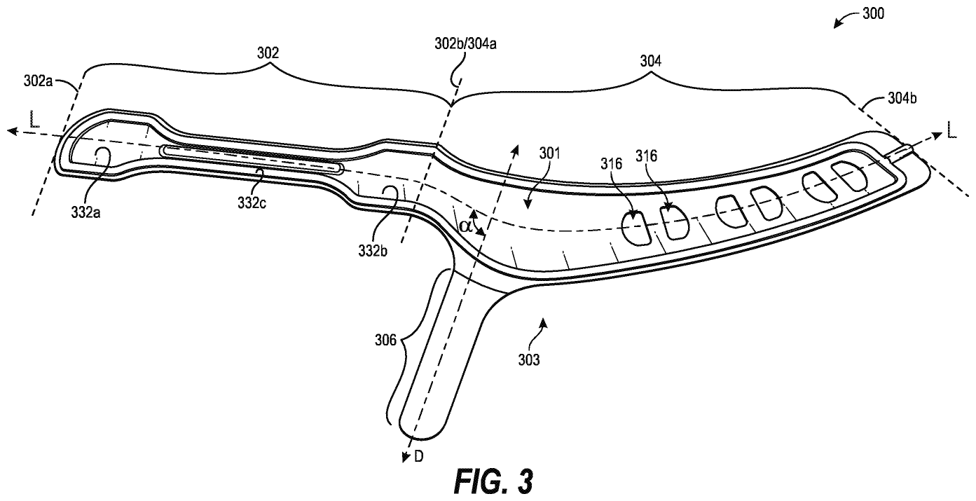

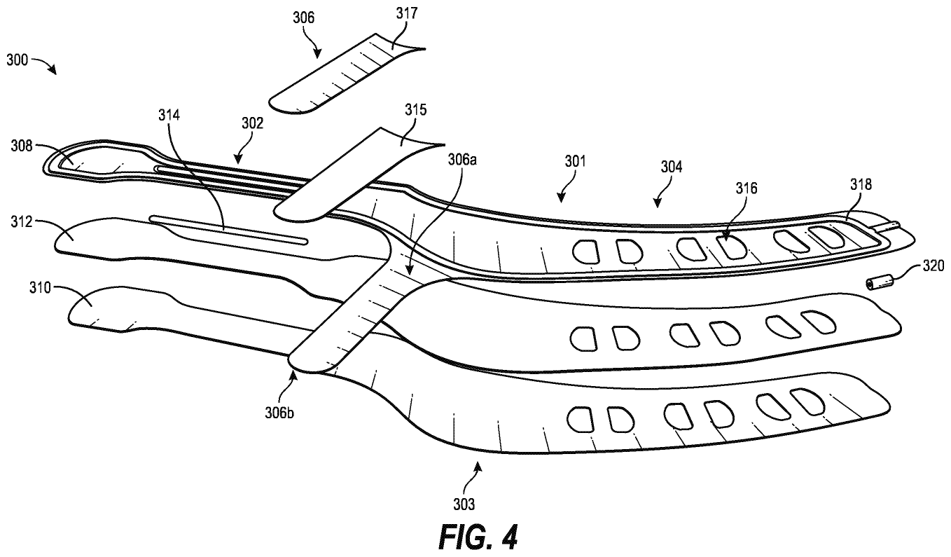

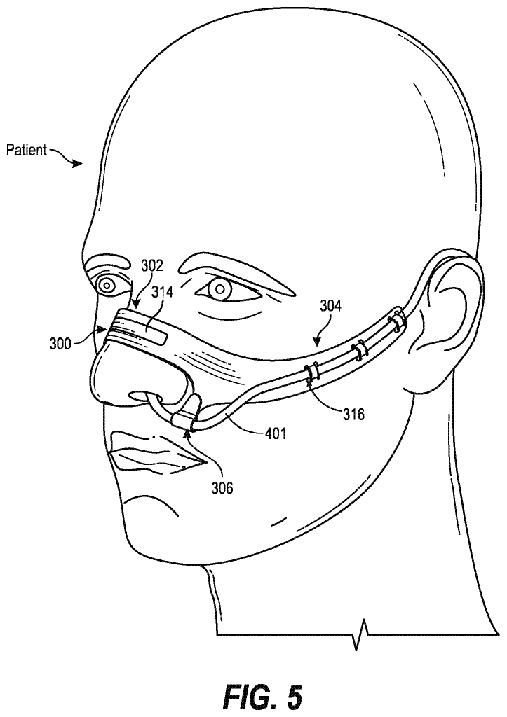

FIGS. 3 and 4 are a top view and an exploded view, respectively, of a securing device 300 (or “device 300”) of the present technology configured to secure an extracorporeal portion of a nasally-inserted, elongated treatment member relative to a face of a patient. FIG. 5 depicts a device similar to device 300 adhered to the patient's face and securing a treatment member 401 thereto. It will be appreciated that the device 300 may be used to secure various types of treatment devices to the patient. Such treatment devices may be insertable through the nasal and/or oral cavity (e.g., feeding tubes, stimulation devices, endoscopes, etc.), through the vasculature, and/or subcutaneously. The device 300 or one or more portions of the device 300 may comprise a large surface area configured to be adhered to the patient's face. The device 300 may further include a high tack adhesive for bonding to the patient's skin. As discussed herein, when the device 300 is adhered to the patient's skin, the large surface area and/or adhesion strength creates significant resistance to removal of the device 300 designed to cause sufficient discomfort that disincentivizes patients from attempting to remove the securing device 300 and/or treatment member and/or prevent the patient from removing the securing device 300.

With reference to FIGS. 3-5, the device 300 comprises a first portion 302 configured to be positioned on and adhered to the patient's nose, a second portion 304 configured to be positioned on and adhered to the patient's cheek and/or jaw, and a third portion 306 extending away from the first and second portions 302, 304 and configured to secure the elongated treatment member 401 to the device 300. In some embodiments, the device 300 does not comprise a third portion 306, and in some embodiments the device comprises two or more third portions 306. The device 300 has a top side 301 configured to face away from the patient's face when the device 300 is adhered to the patient, and a bottom side 303 configured to be positioned on or adjacent the patient's skin. The device 300 further comprises a longitudinal dimension L extending along the first and second portions 302, 304, a width dimension extending between the longitudinal sides of the first and second portions 302, 304, and a thickness dimension measured along a plane perpendicular to the longitudinal dimension L (FIG. 3) between the top and bottom sides of the device 300.

As best shown in FIG. 4, the device 300 may comprise one or more layers disposed along a thickness of the device 300. The one or more layers can comprise a first layer 308 at the top side 301, a second layer 310 at the bottom side 303, and an adhesive element 312 between the first and second layers 308, 310. In some embodiments, the adhesive element 312 extends along only the bottom side 303 of the first and second portions 302, 304 and not along the bottom side 303 of the third portion 306, as the third portion 306 is not intended to be adhered to the patient's skin. In some embodiments, the first layer 308 comprises a fabric or otherwise breathable material and the second layer 310 comprises a backing paper configured to be removed from the device 300 to expose the adhesive element 312 prior to adhering the device 300 to the patient's skin. The first and second layers 308, 310 can additionally or alternatively include other materials.

In some embodiments, the adhesive element 312 comprises an absorbent material with an adhesive disposed on one or more sides of the absorbent material. In some embodiments, the absorbent material is impregnated with the adhesive, and in some embodiments the adhesive can comprise a solid layer adhered to the absorbent material. According to several aspects of the technology, the adhesive is disposed on the absorbent material via spray coating, dip coating, thin film deposition, or another suitable process. In some embodiments, the adhesive element 312 does not comprise an absorbent material. In these and other embodiments, the adhesive can be applied directly to the first layer 308 via spray coating, dip coating, thin film deposition, etc.

As shown in FIG. 3, the first portion 302 of the device 300 can comprise a first terminus 302a, a second terminus 302b, and a length that extends along a first dimension that runs through the first and second termini 302a, 302b. The first portion 302 may comprise a first end region 332a configured to be positioned on and adhered to a first ala region and/or sidewall of the patient's nose, a second end region 332b configured to be positioned on and adhered to a second ala region and/or sidewall of the patient's nose, and an intermediate region 332c extending between the first and second end regions 332a, 332b. The intermediate region 332c is configured to be positioned on and adhered to a dorsum nasi region and/or sidewall of the patient's nose. In some embodiments, the first and second end regions 332a, 332b have a width and/or surface area that is greater than a width of the intermediate region 302c. In these and other embodiments, the intermediate region 332c can have a length greater than a length of each of the first and second ends regions 332a, 332b. According to some embodiments a width of the intermediate region 332c is generally constant along a length of the intermediate region and/or an entire length of the first portion 302.

As shown in FIG. 4, in some embodiments the device 300 includes a resilient member 314 positioned along all or a portion of the length of the first portion 302. For example, the resilient member 314 may be positioned along and/or coincide with all or a portion of the length of the intermediate region 332c. The resilient member 314 may be configured to conform the first portion 302 to a shape of the patient's nose. The resilient member 314, for example, can be positioned between the first layer 308 and the adhesive element 312. The resilient member 314 can comprise a spring, wire, coil, braid, etc. In some embodiments, the resilient member 314 is configured to pull outwardly on the patient's nose to expand one or more of the patient's nasal passages. Such expansion can facilitate insertion of a treatment member into the nasal passage, breathing through the nasal passage, etc.

The second portion 304 of the device 300 can comprise a first terminus 304a, a second terminus 304b, and a length that extends along a second dimension that runs through the first and second termini 304a, 304b. In some embodiments, the first terminus 304a of the second portion 304 corresponds with the second terminus 302b of the first portion 302. In some embodiments, the first terminus 304a of the second portion 304 and the second terminus 302b of the first portion 302 are spaced apart along the longitudinal dimension L of the device 300. The second portion 304 can extend between the first and second termini 304a, 304b along a curved or otherwise indirect path (see FIG. 3) or along a substantially straight path.

The second portion 304 may comprise a greater surface area than that of the first portion 302 and is configured to be adhered to a portion of the patient's face, such as the patient's cheek and/or jaw, with enough adhesive strength to cause discomfort if a patient attempts to prematurely remove the device 300. In some embodiments, the second portion 304 may include one or more slits or openings 316 spaced apart along a longitudinal dimension of the device 300 that are configured to receive a treatment member therethrough. As shown in FIGS. 3 and 4, the one or more openings 316 may be arranged into multiple pairs of openings 316 spaced apart from one another along a length of the second portion 304. In use, for example as shown in FIG. 5, a portion of the treatment member 401 can be threaded through the openings 316 such that at least a portion of the length of the treatment member 401 extends along and is secured to at least a portion of the length of the second portion 304. As such, the device 300 is configured to reduce the amount of slack or unsecured sections of the treatment member 401 for the patient to grab hold of when attempting to remove the device 300. In some embodiments, the second portion 304 does not include any openings and mainly functions to increase the surface area of the device 300 adhered to the patient's skin.

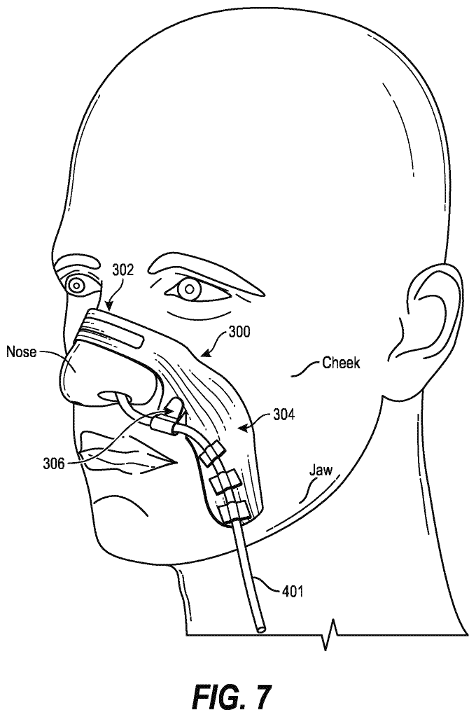

The shape, length, and/or width of the second portion 304 may depend on a desired surface area and/or desired adhesion properties of the device 300. Moreover, an angle between the second dimension of the second portion 304 and the first dimension of the first portion 302 can determine the path along which the second portion 304 extends on the patient's face. The path may be dictated by the stiffness of the treatment member 401 to be secured. For example, stiffer treatment members may require a greater angle between the first and second dimensions than the angle achievable for more flexible treatment members. This is because stiffer treatment members may not have the flexibility to make a sharp turn towards the ear once exiting the nostril (as shown in FIG. 5), and thus must take a more inferiorly-oriented path (as shown in FIG. 7). An electrical stimulation device, for example, has more internal components and thus a greater stiffness than a standard nasogastric feeding tube.

When the angle between the first and second dimensions is lower (e.g., for use with a more flexible treatment member), the second portion 304 is configured to extend away from the first portion 302 in a predominantly lateral direction, for example as shown in FIG. 5. In such embodiments, a first end region of the second portion 304 (including first terminus 304a) can be positioned on and adhered to the patient's nose, such as at or along an infraorbital region, and a second end region of the second portion 304 (including second terminus 304b) can be positioned on and adhered to a side of the patient's face, such as at a zygomatic region, parotid region, and/or buccal region.

When the angle between the first and second dimensions is greater (e.g., for use with a stiffer treatment member), the second portion 304 is configured to extend away from the first portion 302 in a predominantly inferior direction, for example as shown in FIG. 7. In such embodiments, a first end region of the second portion 304 (including first terminus 304a) can be positioned on and adhered to the patient's nose, such as at or along an infraorbital region, and a second end region of the second portion 304 (including second terminus 304b) can be positioned on and adhered to a more anterior region of the patient's face, such as a mental region, oral region, and/or buccal region.

The third portion 306 of the device 300 can comprise a first terminus, a second terminus, and a length that extends along a third dimension D that runs through the first and second termini of the third portion 306. The third portion 306 may extend laterally away from the first and/or second portions 302, 304. In some embodiments, the third portion 306 has a first end region 306a at the first portion 302, the second portion 304, and/or the junction between the first and second portions 302, 304. The third dimension can be disposed at a non-90 degree angle α relative to the first dimension, the second dimension, and/or the longitudinal dimension L of the device 300. In some embodiments, the third portion 306 extends away from the longitudinal dimension L of the first and second portions 302, 304 at an angle α of substantially 90 degrees. As shown in FIG. 3, when the device 300 is adhered to the patient's face but before the third portion 306 is in a secured position, the third portion 306 can extend laterally away from the second portion 304 in an inferior direction.

The third portion 306 can comprise a first layer 308, a second layer 317, and an adhesive element 315 between the first and second layers 308, 317. The first layer 308 may comprise the bottom layer and the second layer 317 may comprise the top layer. As with the first and second portions 302, 304, the first layer 308 can comprise a breathable material and the second layer 317 can comprise a backing paper configured to be removed from the device 300 to expose the adhesive element 315 prior to adhering the third portion 306 to the device 300, the treatment element, and/or the patient's skin. The adhesive element 315 can comprise an absorbent material with an adhesive disposed on one or more sides of the absorbent material. In some embodiments, the absorbent material is impregnated with the adhesive, and in some embodiments the adhesive can comprise a solid layer adhered to the absorbent material. According to several aspects of the technology, the adhesive is disposed on the absorbent material via spray coating, dip coating, deposition, or another suitable process. In some embodiments, the second adhesive element 315 does not comprise an absorbent material. Instead, the adhesive can be applied directly to the first layer 308 and/or the second layer 317.

As previously mentioned, the third portion 306 is configured to secure the elongated treatment member to the device 300. In some embodiments, for example as shown in FIG. 5, the second end region 306b of the third portion 306 may be configured to fold over a length of the extracorporeal portion of the elongated treatment member 401 and adhere to another region of the third portion 306, another portion of the device 300, the treatment member 401, and/or the patient's skin. When the first and second portions 302, 304 are adhered to the patient's face and the third portion 306 has not yet been folded over the elongated treatment member 401, the second layer 317 of the third portion 306 can face away from the patient's face. To secure the elongated treatment member 401 to the device 300, the second layer 317 can be removed from the third portion 306 to expose the adhesive element 315, the length of the extracorporeal portion of the elongated treatment member 401 can be adhered to the adhesive element 315 of the third portion 306, and the third portion 306 can be folded towards the longitudinal dimension L of the device 300 to adhere the third portion 306 to itself, another portion of the device 300, the treatment member 401, and/or the patient's skin.

Additionally or alternatively, the third portion 306 may comprise a non-adhesive-based mechanism of reversibly securing (a) the elongated treatment member 401 to the third portion 306 and/or (b) the third portion 306 to the device 300. For example, in some embodiments the third portion 306 includes a fastener configured to be detachably coupled to a complementary receiving element and/or mating portion of the device 300 (including on the first and/or second portion 302, 304). In some embodiments, the third portion 306 may be configured to loop around and/or be tied around an opening or other retention structure coupled to or integral with the device 300. For example, the first and/or second portion 302, 304 can include an opening configured to receive a portion of the third portion 306 therethrough. According to several embodiments, the device 300 does not include a third portion 306 and instead includes a fastening assembly coupled to the device 300 (such as the first and/or second portion 302, 304). The fastening assembly (not shown) may include a first element configured to engage the treatment member 401 and a second element configured to be releasably coupled to the device 300 (such as to the first and/or second portion 302, 304). The fastening assembly may comprise, for example, a button and loop, magnets, a snap, a clip, etc. In some embodiments, the device 300 includes one or more third portions 306 and one or more fastening assemblies.

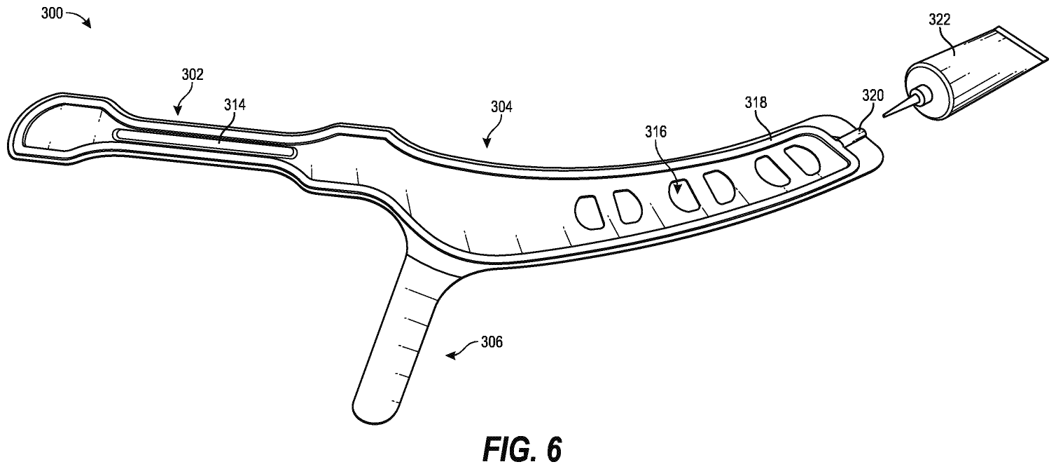

In some embodiments of the present technology, the device 300 can comprise a channel 318 extending along at least a portion of the first portion 302 and/or at least a portion of the second portion 304. For example, as shown in FIG. 4, the channel 318 can extend along the first and second portions 302, 304 at or adjacent a periphery of the first and second portion 302, 304. In some embodiments, the portion(s) and/or material(s) of the device 300 defining the channel 318 do not include the adhesive. For example, the first layer 308 of the first and second portions 302, 304 can define the channel 318 (and not the adhesive element 312). The channel 318 can be configured to receive a releasing agent therethrough to enable mechanical separation of the device from the patient's skin. In some embodiments, the device 300 comprises a valve 320 fluidly coupled to the channel 318 and configured to receive the releasing agent. As shown in FIG. 6, the valve 320 can be configured to receive the releasing agent from an external source 322. The external source 322 can comprise a bottle, a syringe, an aerosol canister, or another suitable dispensing device. In some embodiments, the device 300 does not include a channel and/or valve.

The releasing agent can be configured to interact with the adhesive to degrade the adhesive bond strength between the device 300 and the patient's skin such that the device 300 can be removed from the patient's face without substantial discomfort to the patient. In some embodiments, the releasing agent comprises a medical-grade adhesive remover. The releasing agent can be water-based or solvent-based. The channel 318 can be configured to distribute the releasing agent along a length of the device 300. In embodiments in which one or more portions of the device 300 comprises an absorbent material, the absorbent material can absorb the releasing agent and the releasing agent can diffuse through the absorbent material to regions of the device 300 that are not in direct contact with the channel 318.

In some embodiments, a releasing agent is not required to mechanically separate the device from the patient's face. For example, the adhesive can comprise a light-sensitive adhesive that degrades when exposed to light. In such embodiments, the device can comprise one or more opaque materials configured to be positioned between a light source and the adhesive material in the device. The one or more opaque materials can be configured to be removed from the device when the device is intended to be removed from the patient's face. For example, the first material as disclosed herein can comprise an opaque layer that is configured to be separated from the adhesive element. Accordingly, when the opaque layer is removed from the device, the adhesive element can be exposed to light and can degrade to a sufficient degree such that the device can be mechanically separated from the patient's face.

In some embodiments, the device may comprise a first or top layer and a second or bottom layer. The top layer may include an adhesive on its underside configured to adhere to the bottom layer. The bottom layer may include an adhesive on its underside configured to adhere the bottom layer and/or device to the patient. When the device is adhered to the patient's skin and the top layer is removed (with or without the treatment member attached), the portion of the device remaining on the patient is the bottom layer. The bottom layer may be transparent or otherwise configured to pass light therethrough such that, over time, light passing through the bottom layer inactivates the adhesive to allow the device to be removed.

According to some embodiments, a method of securing an extracorporeal portion of a nasally-inserted elongated treatment member to a device, and thereby a face of a patient, can comprise adhering a nasal portion (e.g., first portion 302 as described herein) of the device to a nose of the patient. In some embodiments, adhering the nasal portion of the device to the patient's nose comprises removing a backing material (e.g., second layer 310 as described herein) from the nasal portion to expose an adhesive and positioning the nasal portion adjacent the patient's nose. In some embodiments, adhering the nasal portion to the nose comprises applying a pressure to the nasal portion once the nasal portion is positioned adjacent the patient's nose. The method can also comprise conforming a resilient member of the nasal portion to the patient's nose.

The method can comprise removing a backing material (e.g., second layer 310 as described herein) from a securing portion (e.g., third portion 306 as described herein) of the device. A length of the extracorporeal portion of the nasally-inserted elongated treatment member can be positioned adjacent an adhesive of the securing portion and the securing portion can be folded over the length of the extracorporeal portion of the nasally-inserted elongated treatment member and adhered to the device. In some embodiments, the securing portion comprises an adhesive such that the length of the extracorporeal portion of the nasally-inserted elongated treatment member is adhered to the securing portion and the securing portion is adhered to the device.

In some embodiments, the method comprises positioning a second length of the extracorporeal portion of the nasally-inserted elongated treatment member through one or more openings defined by a facial portion (e.g., second portion 304 as described herein) of the device so that at least a portion of the elongated treatment member extends along a longitudinal dimension of the device. The second length of the extracorporeal portion of the nasally-inserted elongated treatment member can be inserted through multiple openings such that a greater length of the portion of the elongated treatment member extends along a longitudinal dimension of the device. In some embodiments, a backing material (e.g., second layer 310 as described herein) can be removed to expose an adhesive and the facial portion can be positioned adjacent to and adhered to the patient's cheek. In some embodiments, adhering the facial portion to the cheek comprises applying a pressure to the facial portion once the facial portion is positioned adjacent the patient's cheek.

According to some embodiments, a method of removing a device as described herein from a patient's face to remove an elongated treatment member from the patient can comprise applying a releasing agent to the device. In some embodiments, a releasing agent is delivered from an external source to a valve of the device. The releasing agent can be delivered from the valve to a channel in the device, and the releasing agent can be delivered to one or more locations away from the valve via the channel. In some embodiments, the device comprises one or more absorbent materials configured to deliver the releasing agent to one or more locations away from the channel. The releasing agent can interact (e.g., degrade, dissolve, swell, etc.) with the adhesive to reduce an adhesive bond strength between the device and the patient's face such that the device can be mechanically separated from the patient's face.

### CONCLUSION

Although many of the embodiments are described above with respect to devices, systems, and methods for securing an elongated treatment member to a face of a patient, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

As used herein, the terms “generally,” “substantially,” “about,” and similar terms are used as terms of approximation and not as terms of degree, and are intended to account for the inherent variations in measured or calculated values that would be recognized by those of ordinary skill in the art.

1. A device configured to secure a position of an extracorporeal portion of an elongated treatment member inserted into a body lumen of a patient, the device comprising:
a first portion configured to be adhered to a nose of the patient;
a second portion extending from the first portion along a longitudinal dimension of the device, wherein the second portion is configured to be adhered to a cheek of the patient; and
a third portion extending from the second portion in a direction laterally away from the longitudinal dimension of the device, wherein the third portion is configured to be folded over a length of the extracorporeal portion of the elongated treatment member and secured to the device such that the elongated treatment member is secured to the device,
wherein each of the first and second portions includes an adhesive, and
wherein each of the first, second, and third portions comprise a unitary structure.

2. The device of claim 1, wherein the adhesive has an adhesion strength sufficiently large to prevent the device from being mechanically removed from the patient's face without discomfort.

3. The device of claim 2, wherein, when a releasing agent is applied to the device, the releasing agent reduces the adhesion strength of the adhesive such that the device can be mechanically removed from the patient's face.

4. The device of 1, further comprising a first material and a second material, wherein the first and second materials are coextensive with the first and second portions, wherein the adhesive is positioned between the first and second materials.

5. The device of 4, wherein the first material is configured to face away from the patient's face when the device is adhered to the patient's face, and wherein the second material is configured to be removed prior to adhering the device to the patient's face.

6. The device of claim 1, wherein:
the third portion comprises a first side and a second side opposite the first side along a thickness of the third portion, and, when the first and second portions are adhered to the patient's face and the third portion has not yet been folded over the elongated treatment member, the second side is positioned proximate the patient's face,
wherein the device further comprises a removable element and an adhesive element positioned on the first side of the third portion, wherein the adhesive element is positioned between the removable element and the first side.

7. The device of 1, wherein the second portion of the device comprises one or more openings configured to receive the elongated treatment member such that the elongated treatment member extends along the longitudinal dimension of the device.

8. The device of claim 1, further comprising a channel extending along at least a portion of the first portion and at least a portion of the second portion, wherein the portion of the device defining the channel does not include the adhesive, and wherein the channel is configured to receive a releasing agent therethrough to enable mechanical separation of the device from the patient's skin.

9. The device of claim 8, further comprising a valve fluidly coupled to the channel and configured to receive the releasing agent from an external source.

10. The device of claim 1, wherein the adhesive is a high tack adhesive.

11. A device configured to secure an extracorporeal portion of an elongated treatment member inserted into a body lumen of a patient to a face of a patient, the device comprising:
a nasal portion comprising a first surface including an adhesive and a second surface opposite the first surface along a thickness of the nasal portion, the first surface of the nasal portion being configured to be adhered to a dorsum nasi of the patient, wherein the nasal portion has a first end configured to be adhered to a first ala region of the patient and a second end configured to be adhered to a second ala region of the patient;
a facial portion comprising a first surface including an adhesive and a second surface opposite the first surface along a thickness of the facial portion, the first surface of the facial portion being configured to be adhered to a cheek of the patient, wherein the facial portion has a first end at the nasal portion and a second end spaced apart from the first end along a first direction; and
a securing portion comprising a first surface and a second surface opposite the first surface along a thickness of the securing portion, wherein the securing portion has a first end at the facial portion and a second end spaced apart from the first end along a second direction disposed at an angle with respect to the first direction,
wherein, the securing portion comprises a first state in which the first surface is configured to be positioned proximate the patient's face and a second state in which the securing portion is configured to be folded over the extracorporeal portion of the elongated treatment member such that the second surface is secured to the device and a position of the extracorporeal portion of the elongated treatment member is fixed with respect to the device, and
wherein the second surface of the nasal portion, the second surface of the facial portion, and the first surface of the securing portion comprise a monolithic structure.

12. The device of 11, wherein, when the nasal portion is adhered to the patient's dorsum nasi and the facial portion is adhered to the patient's cheek, an adhesive bond strength between the device and the patient's face prevents the device from being mechanically separated from the patient's face without discomfort to the patient.

13. The device of claim 12, wherein, when a releasing agent is applied to the device, the adhesive bond strength between the device and the patient's face is reduced such that the device can be mechanically separated from the patient's face without discomfort to the patient.

14. The device of claim 11, wherein, when the device is adhered to the patient's face, the first direction extends from the first end to the second end along a substantially inferior direction.

15. The device of 11, wherein the elongated treatment member is at least one of a feeding tube, an endoscope, or an electrical stimulation device.

16. The device of claim 11, wherein at least one of the first surface of the nasal portion, the first surface of the facial portion, or the second surface of the securing portion comprises a backing material configured to prevent the device from being adhered to the patient's face until the backing material is removed.

17. The device of claim 11, wherein at least one of the first surface of the nasal portion, the first surface of the facial portion, or the second surface of the securing portion comprises an absorbent material carrying the adhesive.

18. A device configured to secure a position of a proximal portion of an elongated treatment member inserted into a body lumen of a patient, the device comprising:
a first broad surface comprising a first shape including a first portion, a second portion, and a third portion; and
a second broad surface opposite the first broad surface along a thickness of the device, the second broad surface comprising a second shape including a first portion, a second portion, and a third portion, wherein the second shape is substantially the same as the first shape,
wherein the first and second portions of the first broad surface and the third portion of the second broad surface comprise an absorbent material with an adhesive disposed thereon,
wherein the third portion of the first broad surface and the first and second portions of the second broad surface comprise a breathable material,
wherein the first and second portions of the first broad surface are configured to be adhered to a nose of the patient and a face of the patient, respectively, and
wherein the third portion of the second broad surface is configured to be adhered to the proximal portion of the elongated treatment member.

19. The device of claim 18, wherein the first and second portions of the first broad surface have a surface area sufficiently large to prevent the patient from prematurely removing the device from the patient's face.

20. The device of claim 18, wherein the device is configured to receive a solvent configured to dissolve the adhesive such that the device can be removed from the patient's face without discomfort to the patient.

21. The device of claim 18, wherein the breathable material is opaque and the adhesive is light-sensitive.

22. The device of claim 18, wherein the breathable material is configured to be removed from the device such that the adhesive is exposed to light and dissolves such that the device can be removed from the patient's face without discomfort to the patient.

23-25. (canceled)
